(19) United States
(12) Patent Application Publication
(10) Pub. No.: US 2009/0143771 A1
(43) Pub. Date: Jun. 4, 2009

(54) FILTER REMOVAL DEVICE

(75) Inventors: Brendan Casey, Barna (IE); Gerard Rabbitte, Tuam (IE)

Correspondence Address: Sughrue Mion-ABBOTT LABS, 2100 Pennsylvania Avenue, N.W., Washington, DC, 20037, US

(73) Assignee: SALVIAC LIMITED, Dublin (IE)

(21) Appl. No.: 12/256,548

(22) Filed: Oct. 23, 2008

Related U.S. Application Data
(60) Provisional application No. 60/983,172, filed on Oct. 27, 2007.

(51) Int. Cl. A61B 17/00

(52) U.S. Cl. 606/1

(57) ABSTRACT

A device for removing a filter element from a retrieval catheter is provided. The device includes a core member which has a diameter corresponding to a filter element; a handle at a proximal end of the core member; and an engagement portion, formed at a distal end of the core member, for engaging the filter element. Alternatively, the device includes a first member including a core wire, a core wire handle; and an engagement portion, and a second member including a hypotube; and a hypotube handle, wherein the core wire is moveably disposed coaxially inside of the hypotube, such that when a distal end of the core wire handle is pressed against a proximal end of the hypotube handle, the engagement portion of the first member extends beyond a distal end of the hypotube. Alternatively, the device includes a partially cylindrical member, a handle; and at least one blade.

### BACKGROUND OF THE INVENTION

1. Field of the Invention

Devices consistent with the present invention relate to tools for use with catheters and, more particularly, to tools for removing filters and other devices from retrieval catheters.

2. Description of the Related Art

In the case of treating the buildup of plaque or other occlusions in the carotid artery, a related art procedure commonly uses a filter element which is deployed downstream of a treatment area, i.e., between the treatment area and the brain, in order to filter and remove any pieces of plaque or occlusion material which may be dislodged during treatment and thus enter the brain causing a stroke or other damage. U.S. Patent Application Publication No. 2002/0049467, which is herein incorporated by reference in its entirety, describes an example of such a related art procedure. U.S. Pat. No. 6,336,934, which is herein incorporated by reference in its entirety, describes examples of related art filter elements.

First, a delivery catheter is loaded with the filter element, inserted through the leg and threaded, typically using a guidewire, beyond a treatment region, where the filter element is deployed such that the filter element is downstream of the treatment region. The desired treatment is then carried out, and the filter element filters any dislodged plaque material. Then, a retrieval catheter is inserted through the leg and threaded through the vasculature to the treatment region, and the filter element is retrieved into the retrieval catheter, and the retrieval catheter, along with the filter element, is removed from the body.

Once the retrieval catheter, along with the filter element inside, is removed from the patient, it is often desirable for a physician to be able to harvest the plaque or occlusion material present in the filter element in order to better understand the nature and/or composition of the material, or to otherwise analyze the material in order to produce more effective drugs for use in treatment. However, it is difficult to remove the filter element from the body of the retrieval catheter due to, for example, the geometry and small size of the filter element and the geometry and small size of the retrieval catheter tip.

### SUMMARY OF THE INVENTION

Exemplary embodiments of the present invention address the above disadvantages and other disadvantages not described above. Also, the present invention is not required to overcome the disadvantages described above.

An object of the present invention is to provide a device which may easily remove a filter element from a retrieval catheter.

According to an aspect of the present invention, there is provided a device for removing a filter element from a retrieval catheter, the device including a core member which has a diameter corresponding to a filter element which is to be removed from the retrieval catheter; a handle at a proximal end of the core member; and an engagement portion, formed at a distal end of the core member, for engaging the filter element.

According to another aspect of the present invention, there is provided A device for removing a filter element from a retrieval catheter, the device including a first member including a core wire which has a diameter corresponding to a filter element which is to be removed from the retrieval catheter; a core wire handle at a proximal end of the core wire; and an engagement portion, formed at a distal end of the core wire, for engaging the filter element; and a second member including a hypotube; and a hypotube handle formed around a proximal end of the hypotube, wherein the core wire is moveably disposed coaxially inside of the hypotube, such that when a distal end of the core wire handle is pressed against a proximal end of the hypotube handle, the engagement portion of the first member extends beyond a distal end of the hypotube.

According to yet another aspect of the present invention, there is provided a device for removing a filter element from a retrieval catheter, the device including a partially cylindrical member having an inner diameter corresponding to an outer diameter of a retrieval catheter; a handle disposed on the outside wall and parallel to the axis of the partially cylindrical member; and at least one blade disposed on an inside wall and parallel to the axis of the partially cylindrical member.

### BRIEF DESCRIPTION OF THE DRAWINGS

The above and other aspects of the present invention will become more apparent by describing in detail exemplary embodiments thereof with reference to the attached drawings, in which:

FIG. 1 is a view showing a device for removing a filter element from a retrieval catheter according to a first exemplary embodiment of the present invention;

FIG. 2A is a view showing a device for removing a filter element from a retrieval catheter according to a second exemplary embodiment of the present invention;

FIG. 2B is a close-up view of detail A of a distal end of the device of FIG. 2A;

FIG. 3A is a view showing a device for removing a filter element from a retrieval catheter according to a third exemplary embodiment of the present invention;

FIG. 3B is a close-up cross-sectional view of a distal end of the device of FIG. 3A;

FIG. 4A is a view showing a device for removing a filter element from a retrieval catheter according to a fourth exemplary embodiment of the present invention;

FIG. 4B is a close-up view of detail B of a distal end of the device of FIG. 4A;

FIG. 5A is a view showing a device for removing a filter element from a retrieval catheter according to a fifth exemplary embodiment of the present invention;

FIG. 5B is a close-up view of a distal end of the device of FIG. 5A;

FIG. 6A is a view showing a device for removing a filter element from a retrieval catheter according to a sixth exemplary embodiment of the present invention;

FIG. 6B is a close-up view of detail C of a distal end of the device of FIG. 6A;

FIG. 7A is a view showing a device for removing a filter element from a retrieval catheter according to a seventh exemplary embodiment of the present invention; and

FIG. 7B is a top view of the device of FIG. 7A.

### DESCRIPTION OF EXEMPLARY EMBODIMENTS OF THE PRESENT INVENTION

Hereinafter, exemplary embodiments of the present inventive concept will be described in detail with reference to the drawings. The same reference numbers are used to denote the same elements in different drawings.

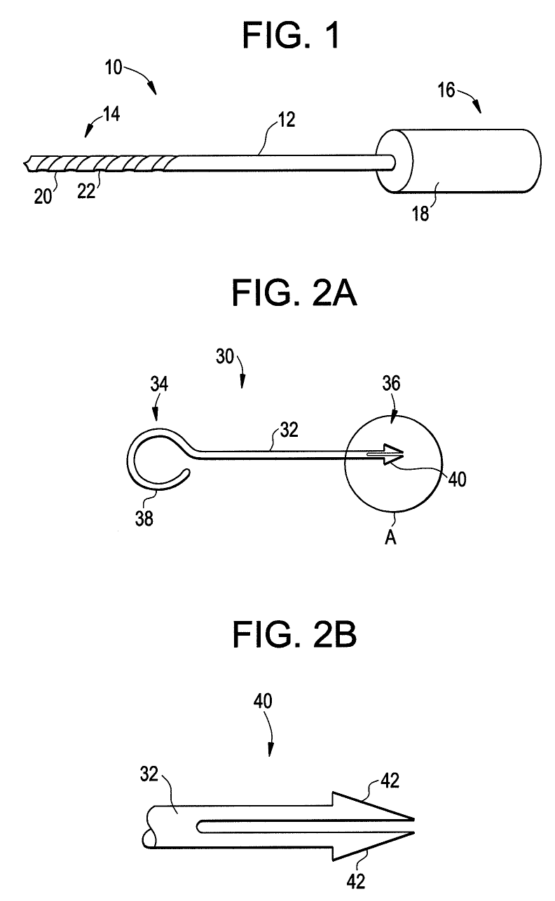

Turning to FIG. 1, a device for removing a filter element from a retrieval catheter according to a first exemplary embodiment of the present invention is shown. The device 10 includes a core member 12 having a distal end 14 and a proximal end 16. The distal end 14 has an engagement portion 20 having a plurality of threads 22 formed in the core member 12. The threads may be of a uniform pitch. The core member 12 may be a core wire. At the proximal end 16 of the core member 12 is disposed a handle 18. The handle 18 may be a cylindrical grip. The diameter of the core member 12 is selected such that the diameter is slightly larger than an inner diameter of a filter element which will be removed.

A user grasps the handle 18 and inserts the distal end 14 of the device 10 into a distal end of a retrieval catheter which has a retrieved filter element stored therein. The core member 12 is aligned with the inner diameter of the filter element, and pressure is applied while the handle 18 is twisted so as to screw the engagement portion 20 into the end of the filter element stored in the retrieval catheter to engage the filter element. The handle 18 is then pulled in a proximal direction to pull the engaged filter element out of the distal end of the retrieval catheter. Thus, the filter element is removed from the retrieval catheter.

Turning to now to FIGS. 2A and 2B, a device for removing a filter element from a retrieval catheter according to a second exemplary embodiment of the present invention is shown. The device 30 includes a core member 32 having a distal end 36 and a proximal end 34. The distal end 36 has an engagement portion 40 having at least two barbed tines 42 formed at the distal end of the core member 32. The engagement portion 40 may also be a barbed dart. The core member 32 may be a core wire. At the proximal end 34 of the core member 32 is disposed a handle 38. The handle 38 in this exemplary embodiment is a graspable geometric form formed from the core member at the proximal end of the core member. The diameter of the at least two barbed tines is selected such that the diameter is slightly larger than an inner diameter of a filter element which will be removed.

A user grasps the handle 38 and inserts the distal end 36 of the device 30 into a distal end of a retrieval catheter which has a retrieved filter element stored therein. The core member 32 is aligned with the inner diameter of the filter element, and pressure is applied while the handle 38 to insert the engagement portion 40 into the end of the filter element stored in the retrieval catheter to engage the filter element. The barbs of the at least two barbed tines 42 act to engage the filter element. The handle 38 is then pulled in a proximal direction to pull the thus-engaged filter element out of the distal end of the retrieval catheter. Thus, the filter element is removed from the retrieval catheter.

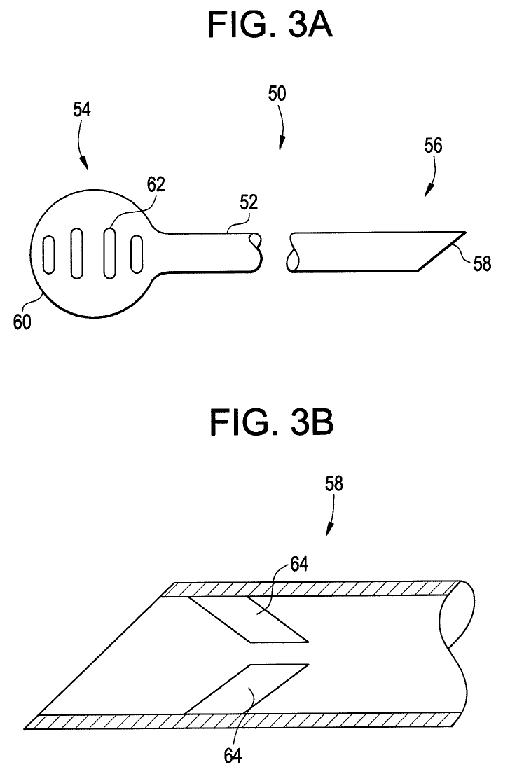

Turning to now to FIGS. 3A and 3B, a device for removing a filter element from a retrieval catheter according to a third exemplary embodiment of the present invention is shown. The device 50 includes a core member 52 having a distal end 56 and a proximal end 54. The distal end 56 of the device 50 has an engagement portion 58. The core member 52 may be a hollow cylindrical tube. At the proximal end 54 of the core member 52 is disposed a handle 60. The handle 60 in this exemplary embodiment is in the form of a paddle and has at least one shallow indentation 62 therein. The distal end 56 of the device 50 at the engagement portion 58 is skived at an angle. The engagement portion 58 includes at least two inverted bars 64 disposed on an inside wall of the core member 52. The at least two inverted bars 64 are spaced a distance from the distal end 56 of the device 50. The outer diameter of the core member 52 is selected so as to match an inner diameter of the retrieval catheter tip.

A user grasps the handle 60 and inserts the distal end 56 of the device 50 into a distal end of a retrieval catheter which has a retrieved filter element stored therein. The indentations 62 in the handle 60 allow for a user to more easily grip the handle 60. The skive at the distal end 56 of the core member 52 helps the core member 52 to be inserted into the retrieval catheter tip and helps direct a neck of the filter into the device 50. The user then slides the distal end 56 of the device 50 into the retrieval catheter until the inverted bars 64 projecting from the inside walls of the core member 52 engage and grip the neck of the filter element. The handle 60 is then pulled in a proximal direction to pull the engaged filter element out of the distal end of the retrieval catheter. Thus, the filter element is removed from the retrieval catheter.

Turning to now to FIGS. 2A and 2B, a device for removing a filter element from a retrieval catheter according to a second exemplary embodiment of the present invention is shown. The device 30 includes a core member 32 having a distal end 36 and a proximal end 34. The distal end 36 has an engagement portion 40 having at least two barbed tines 42 formed at the distal end of the core member 32. The engagement portion 40 may also be a barbed dart. The core member 32 may be a core wire. At the proximal end 34 of the core member 32 is disposed a handle 38. The handle 38 in this exemplary embodiment is a graspable geometric form formed from the core member at the proximal end of the core member. The diameter of the at least two barbed tines 42 is selected such that the diameter is slightly larger than an inner diameter of a filter element which will be removed.

A user grasps the handle 38 and inserts the distal end 36 of the device 30 into a distal end of a retrieval catheter which has a retrieved filter element stored therein. The core member 32 is aligned with the inner diameter of the filter element, and pressure is applied while the handle 38 to insert the engagement portion 40 into the end of the filter element stored in the retrieval catheter to engage the filter element. The barbs of the at least two barbed tines 42 act to engage the filter element. The handle 38 is then pulled in a proximal direction to pull the thus-engaged filter element out of the distal end of the retrieval catheter. Thus, the filter element is removed from the retrieval catheter.

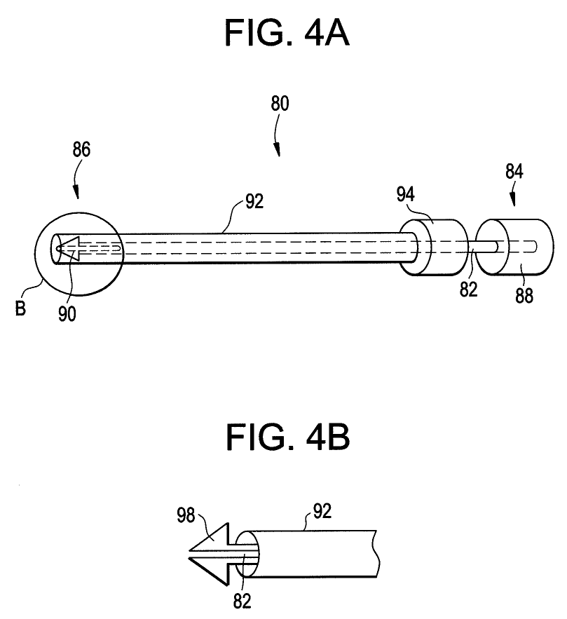

Turning to now to FIGS. 4A and 4B, a device for removing a filter element from a retrieval catheter according to a fourth exemplary embodiment of the present invention is shown. The device 80 includes a first member and a second member. The first member includes a core wire 82 having a distal end 86 and a proximal end 84. The distal end 86 has an engagement portion 90. At the proximal end 84 of the core wire 82 is disposed a core wire handle 88. The core wire handle 88 is a cylindrical grip, and is secured to the core wire 82. The engagement portion 90 has at least two barbed tines 98 at the distal end thereof. The diameter of the at least two barbed tines 98 is selected such that the diameter is slightly larger than an inner diameter of a filter element which will be removed.

The second member includes a hypotube 90 and a hypotube handle 94 disposed at a proximal end thereof. The hypotube handle 94 is also a cylindrical grip, and is formed on the outside wall of the hypotube 92. The hypotube is open at both distal and proximal ends.

The core wire 82 of the first member is movably disposed coaxially in the hypotube 92 of the second member.

A user grasps the core wire handle 88 of the first member and the hypotube handle 94 of the second member, and pushes the core wire handle 88 and the hypotube handle 94 together such that a proximal end of the hypotube handle 94 meets a distal end of the core wire handle 88. This extends the engagement portion 90 of the core wire 82 outside of the distal end of the hypotube 92. When pushed together, the hypotube handle 94 and the core wire handle 88 feel more like a single handle to the user.

Thus combined, the user inserts the distal end of the first and second members of the device 80 into the distal end of the retrieval catheter which has a retrieved filter element stored therein. The user then aligns the barbed tines 98 with the inner diameter of the filter element, and pressure is applied with the combined hypotube handle 94 and core wire handle 88 to insert the engagement portion 90 into the end of the filter element stored in the retrieval catheter to engage the filter element. The barbs of the at least two barbed tines 98 act to engage the filter element.

The hypotube handle 94 is the moved distally from the core wire handle 88, i.e., the hypotube handle 94 and the core wire handle 88 are moved away from each other, such that the distal end of the hypotube 92 slides over the filter element. At the same time, the filter element is pulled into the hypotube 92 by the gripping action of the barbs of the barbed tines 98. Thus, inside the hypotube 92, the device 80, along with the engaged filter element, is removed from the retrieval catheter.

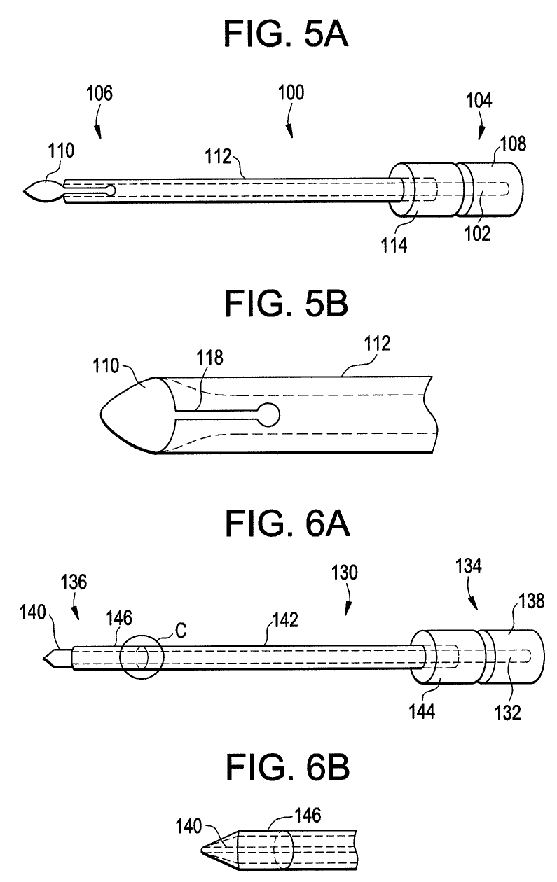

Turning to now to FIGS. 5A and 5B, a device for removing a filter element from a retrieval catheter according to a fifth exemplary embodiment of the present invention is shown. The device 100 includes a first member and a second member. The first member includes a core wire 102 having a distal end 106 and a proximal end 104. The distal end 106 has an engagement portion 110. In this exemplary embodiment, the engagement portion 110 is shown in the form of a bead. At the proximal end 104 of the core wire 102 is disposed a core wire handle 108. The core wire handle 108 is a cylindrical grip, and is secured to the core wire 102. The diameter of the bead of the engagement portion 110 is selected such the bead may be inserted into an end of a filter element such that the bead may engage the filter element by friction.

The second member includes a hypotube 112 and a hypotube handle 114 disposed at a proximal end thereof. The hypotube handle 114 is also a cylindrical grip, and is formed on the outside wall of the hypotube 112. The hypotube 112 is open at both distal and proximal ends. Moreover, the hypotube 112 is provided with at least one slot 118 at a distal end thereof.

The core wire 102 of the first member is movably disposed coaxially in the hypotube 112 of the second member.

A user grasps the core wire handle 108 of the first member and the hypotube handle 114 of the second member, and pushes the core wire handle 108 and the hypotube handle 114 together such that a proximal end of the hypotube handle 114 meets a distal end of the core wire handle 108. This extends the engagement portion 110 of the core wire 102 outside of the distal end of the hypotube 112. When pushed together, the hypotube handle 114 and the core wire handle 108 feel more like a single handle to the user.

Thus combined, the user inserts the distal end of the first and second members of the device 100 into the distal end of the retrieval catheter which has a retrieved filter element stored therein. The user then aligns the bead of the engagement portion 110 with the inner diameter of the filter element, and pressure is applied with the combined hypotube handle 114 and core wire handle 108 to insert the engagement portion 110 into the end of the filter element stored in the retrieval catheter to engage the filter element. The bead of the engagement portion 110 engages the inner diameter of the end of the filter element by friction.

The hypotube handle 114 is the moved distally from the core wire handle 108, i.e., the hypotube handle 114 and the core wire handle 108 are moved away from each other, such that the distal end of the hypotube 112 slides over the filter element. At the same time, the filter element is pulled into the hypotube 112 by the gripping action of the bead of the engagement portion 110. The at least one slot 118 in the distal end of the hypotube 112 permits the distal end of the hypotube 112 to expand to accommodate the filter element. The device 100, along with the engaged filter element, is then removed from the retrieval catheter.

Turning to now to FIGS. 6A and 6B, a device for removing a filter element from a retrieval catheter according to a sixth exemplary embodiment of the present invention is shown. The device 130 includes a first member and a second member. The first member includes a core wire 132 having a distal end 136 and a proximal end 134. The distal end 136 has an engagement portion 140. In this exemplary embodiment, the engagement portion 140 is shown in the form of a dart. At the proximal end 134 of the core wire 132 is disposed a core wire handle 138. The core wire handle 138 is a cylindrical grip, and is secured to the core wire 132. The diameter of the dart of the engagement portion 140 is selected such the dart may be inserted into an end of a filter element such that the dart may engage the filter element by friction.

The second member includes a hypotube 142 and a hypotube handle 144 disposed at a proximal end thereof. The hypotube handle 144 is also a cylindrical grip, and is formed on the outside wall of the hypotube 142. The hypotube 142 is open at both distal and proximal ends. Moreover, the distal end of the hypotube is a rubber sleeve 146 and is tapered.

The core wire 132 of the first member is movably disposed coaxially in the hypotube 142 of the second member.

A user grasps the core wire handle 138 of the first member and the hypotube handle 144 of the second member, and pushes the core wire handle 138 and the hypotube handle 144 together such that a proximal end of the hypotube handle 144 meets a distal end of the core wire handle 138. This extends the engagement portion 140 of the core wire 132 outside of the distal end of the hypotube 142. When pushed together, the hypotube handle 144 and the core wire handle 138 feel more like a single handle to the user.

Thus combined, the user inserts the distal end of the first and second members of the device 130 into the distal end of the retrieval catheter which has a retrieved filter element stored therein. The user then aligns the dart of the engagement portion 140 with the inner diameter of the filter element, and pressure is applied with the combined hypotube handle 144 and core wire handle 138 to insert the engagement portion 140 into the end of the filter element stored in the retrieval catheter to engage the filter element. The dart of the engagement portion 140 engages the inner diameter of the end of the filter element by friction.

The hypotube handle 144 is the moved distally from the core wire handle 138, i.e., the hypotube handle 144 and the core wire handle 138 are moved away from each other, such that the distal end of the hypotube 142 slides over the filter element. At the same time, the filter element is pulled into the hypotube 142 by the gripping action of the bead of the engagement portion 140. The tapered rubber sleeve 146 at the distal end of the hypotube 142 permits the outer diameter of the rubber sleeve to expand or flare when compressed. The device 13-, along with the engaged filter element, is then removed from the retrieval catheter.

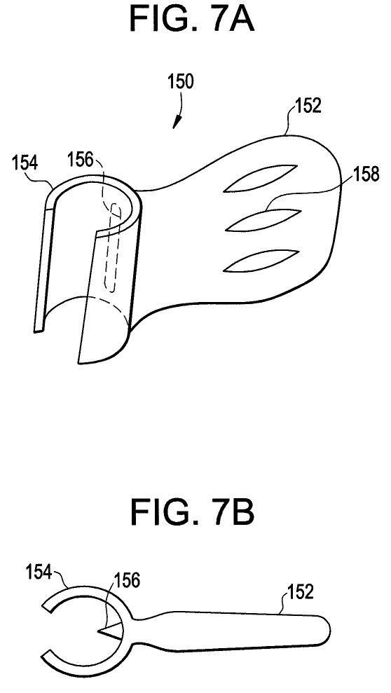

Turning now to FIGS. 7A and 7B, a device for removing a filter element from a retrieval catheter according to a seventh exemplary embodiment of the present invention is shown. The device 150 includes a partially cylindrical member 154 and a handle 152. The partially cylindrical member 154 has an inner diameter which corresponds to an outer diameter of the retrieval catheter. The handle 152 is disposed on the outside wall of the partially cylindrical member 154 and is parallel to the axis of the partially cylindrical member 154. At least one blade 156 is disposed on an inside wall of the partially cylindrical member 154, and is also parallel to the axis of the partially cylindrical member 154. The handle 152 is in the form of a flat plate and has at least one shallow indentation 158 therein. The partially cylindrical member 154 may also be a full cylinder which is hollow.

After a retrieval catheter, containing a filter element, is removed from a patient, a user holds the handle 152 of the device 150 and threads one end of the partially cylindrical member 154 over the tip of the retrieval catheter, so that the axis of the partially cylindrical member 154 is parallel and coaxial to the axis of the retrieval catheter tip. The user then slides the partially cylindrical member 154 along the length of the retrieval catheter tip and the at least one blade 156 slices open the side wall of the retrieval catheter tip, thus exposing the filter element therein.

The at least one blade 156 is sized according to a thickness of the wall of the retrieval catheter tip in order to allow the outside wall of the retrieval catheter tip to be sliced without cutting the filter element therein.

Alternatively, the device 150 may be used in a similar fashion to slice open the outer casing of a filter element itself. In such a case, an inner diameter of the partial cylindrical member 154 is selected to correspond to an outside diameter of a filter element.

The foregoing exemplary embodiments and advantages are merely exemplary and are not to be construed as limiting the present invention. The present teaching can be readily applied to other types of apparatuses. Also, the description of the exemplary embodiments of the present invention is intended to be illustrative, and not to limit the scope of the claims, and many alternatives, modifications, and variations will be apparent to those skilled in the art.

What is claimed is:

1. A device for removing a filter element from a retrieval catheter, the device comprising:
a core member which has a diameter corresponding to a filter element which is to be removed from the retrieval catheter;
a handle at a proximal end of the core member; and
an engagement portion, formed at a distal end of the core member, for engaging the filter element.

2. The device of claim 1, wherein the core member is a core wire.

3. The device of claim 2, wherein the diameter of the core wire is slightly larger than an inner diameter of the filter element which is to be removed.

4. The device of claim 3, wherein the engagement portion is a plurality of threads formed in the core wire at the distal end of the core wire.

5. The device of claim 2, wherein the engagement portion is at least two barbed tines formed at the distal end of the core wire.

6. The device of claim 5, wherein the at least to barbed tines are sized to engage the filter element.

7. The device of claim 2, wherein the engagement portion is a barbed dart formed at the distal end of the core wire.

8. The device of claim 2, wherein the engagement portion is a bead formed at the distal end of the core wire and having a diameter that is slightly larger than the inner diameter of the filter element such that the bead may engage the filter element through friction.

9. The device of claim 1, wherein the handle is a cylindrical grip disposed around the core member at the proximal end of the core member.

10. The device of claim 2, wherein the handle is a section of the proximal end of the core wire which is bent to form a graspable geometric form.

11. The device of claim 1, wherein the handle is a paddle having at least two shallow indentations cut therein.

12. The device of claim 1, wherein the core member is a hollow axial tube, and an outer diameter of the hollow axial tube is equal to an inner diameter of the retrieval catheter.

13. The device of claim 12, wherein the distal end of the hollow axial tube is skived at an angle, and the engagement portion comprises at least two inverted bars formed on an inside wall of the hollow axial tube and spaced a distance from the distal end of the hollow axial tube.

14. A device for removing a filter element from a retrieval catheter, the device comprising:
a first member comprising:
a core wire which has a diameter corresponding to a filter element which is to be removed from the retrieval catheter;
a core wire handle at a proximal end of the core wire; and
an engagement portion, formed at a distal end of the core wire, for engaging the filter element; and
a second member comprising:
a hypotube; and
a hypotube handle formed around a proximal end of the hypotube,
wherein the core wire is moveably disposed coaxially inside of the hypotube, such that when a distal end of the core wire handle is pressed against a proximal end of the hypotube handle, the engagement portion of the first member extends beyond a distal end of the hypotube.

15. The device of claim 14, wherein the engagement portion is at least two barbed tines formed at the distal end of the core wire.

16. The device of claim 14, wherein the engagement portion is a barbed dart formed at the distal end of the core wire.

17. The device of claim 14, wherein the engagement portion is a bead formed at the distal end of the core wire and having a diameter that is slightly larger than the inner diameter of the filter element such that the bead may engage the filter element through friction.

18. The device of claim 17, wherein at least one slot is cut in the distal end of the hypotube to allow the diameter at the distal end of the hypotube to expand.

19. The device of claim 14, wherein a distal end of the hypotube is formed of rubber sleeve having an outer diameter such that the outer diameter expands when compressed.

20. The device of claim 19, wherein the distal end of the hypotube is tapered to allow the rubber sleeve to flare.

21. A device for removing a filter element from a retrieval catheter, the device comprising:
a partially cylindrical member having an inner diameter corresponding to an outer diameter of a retrieval catheter;
a handle disposed on the outside wall and parallel to the axis of the partially cylindrical member; and
at least one blade disposed on an inside wall and parallel to the axis of the partially cylindrical member.

22. The device of claim 21, wherein the partially cylindrical member is a hollow cylinder.

23. The device of claim 21, wherein the partially cylindrical member has walls of a thickness to allow the walls to flex to accommodate the retrieval catheter.

24. The device of claim 21, wherein a size of the at least one blade is selected to be equal to a thickness of an outside wall of the retrieval catheter.
